(19) United States
(12) Patent Application Publication
(10) Pub. No.: US 2023/0057317 A1
(43) Pub. Date: Feb. 23, 2023

(54) METHOD AND SYSTEM FOR AUTOMATICALLY RECOMMENDING ULTRASOUND EXAMINATION WORKFLOW MODIFICATIONS BASED ON DETECTED ACTIVITY PATTERNS

(71) Applicant: GE Precision Healthcare LLC, Wauwatosa, WI (US)

(72) Inventors: Sharath Bhaskar Kadidal, Vöcklabruck (AT); Christian Perrey, Mondsee (AT)

(21) Appl. No.: 17/408,618

(22) Filed: Aug. 23, 2021

(51) Int. Cl. G16H 40/20; G16H 40/63; G06F 3/0484

(52) U.S. Cl. CPC G16H 40/20; G16H 40/63; G06F 3/0484

(57) ABSTRACT

A system and method for automatically recommending ultrasound examination workflow modifications based on detected activity patterns is provided. The method includes analyzing system activity detected during performance of one or more workflows of an ultrasound examination. The method includes detecting a recurring system activity pattern over a plurality of ultrasound examinations. The method includes presenting, at a display system and based on the detecting the recurring system activity pattern, a prompt providing a recommendation to apply a macro to automate a series of system activities in response to detecting a triggering system activity. The method includes receiving a user input accepting the recommendation to apply the macro. The method includes applying the macro to automate the series of system activities in response to the detecting the triggering system activity.

### FIELD

Certain embodiments relate to ultrasound imaging. More specifically, certain embodiments relate to a method and system for automatically recommending ultrasound examination workflow modifications by analyzing activity patterns and suggesting preset modifications, display screen simplification, menu rearrangement, and/or macro generation based on detected recurring activity patterns.

### BACKGROUND

Ultrasound imaging is a medical imaging technique for imaging organs and soft tissues in a human body. Ultrasound imaging uses real time, non-invasive high frequency sound waves to produce a series of two-dimensional (2D) and/or three-dimensional (3D) images.

Ultrasound examinations may include one or more workflows, such as a patient intake workflow, imaging workflow, measurement workflow, analysis workflow, and/or any suitable workflow. Ultrasound system users often repeat many steps in performing the workflow(s) of an ultrasound examination, which may be time consuming. For example, an ultrasound system user may adjust gain and or contrast settings each time a particular preset is selected. As another example, an ultrasound system user may only use a portion of the features presented at a display system of the ultrasound system that is applicable to an ultrasound examination type that the user typically performs. Furthermore, an ultrasound system user may frequently switch between menus or move to sub-menus to access commonly used fields. Moreover, an ultrasound system user may always move images to a disk, move images to cloud storage, print a report, and/or the like at the end of an ultrasound examination. The additional steps to modify settings of a selected preset, navigate menus and sub-menus to frequently used fields, and/or repeat commonly performed tasks may be inefficient. Additionally, the presentation of unused features at a display system may be viewed as clutter and can be visually distracting to an ultrasound system user.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for automatically recommending ultrasound examination workflow modifications based on detected activity patterns, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to automatically recommend ultrasound examination workflow modifications based on detected activity patterns, in accordance with various embodiments.

FIG. 2 is a flow chart illustrating exemplary steps that may be utilized for automatically recommending ultrasound examination workflow modifications based on detected activity patterns, in accordance with various embodiments.

### DETAILED DESCRIPTION

Certain embodiments may be found in a method and system for automatically recommending ultrasound examination workflow modifications based on detected activity patterns. For example, aspects of the present disclosure have the technical effect of analyzing ultrasound system activity (e.g., user interaction, lack of user interaction, automated image view determinations, etc.) to recognize system activity patterns during ultrasound examination workflow(s) over time. Moreover, aspects of the present disclosure have the technical effect of detecting recurring system activity patterns based on analysis of the ultrasound system activity during ultrasound examination workflow(s) over time. Furthermore, aspects of the present disclosure have the technical effect of streamlining preset usage by prompting a user to modify a preset based on detected recurring modifications to the settings of a preset. Additionally, aspects of the present disclosure have the technical effect of simplifying (e.g., decluttering) a display screen by prompting a user to modify feature presentation at a display system of an ultrasound system based on detected patterns of interaction and/or lack of interaction with displayed features. Aspects of the present disclosure have the technical effect of reducing user navigation through menus and submenus by prompting a user to modify menu presentation at a display system of an ultrasound system based on detected menu navigation patterns to access fields within menus and submenus. Also, aspects of the present disclosure have the technical effect of automating ultrasound system actions by prompting a user to generate macros that automate system actions in response to detecting system activity patterns. Furthermore, aspects of the present disclosure have the technical effect of automating ultrasound system actions by applying generated macros that automatically execute a series of system activities in response to a detected triggering activity.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, Contrast Enhanced Ultrasound (CEUS), and/or sub-modes of B-mode and/or CF such as Harmonic Imaging, Shear Wave Elasticity Imaging (SWEI), Strain Elastography, TVI, PDI, B-flow, MVI, UGAP, and in some cases also MM, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphic Processing Unit (GPU), DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

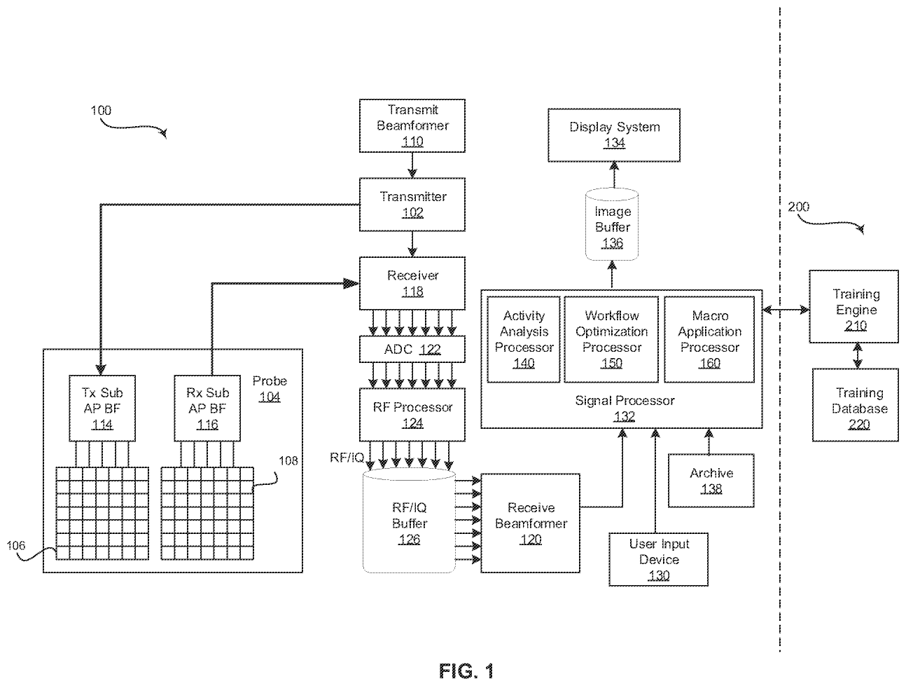

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to automatically recommend ultrasound examination workflow modifications based on detected activity patterns, in accordance with various embodiments. Referring to FIG. 1, there is shown an ultrasound system 100 and a training system 200. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, and an archive 138.

The transmitter 102 may comprise suitable logic, circuitry, interfaces and/or code that may be operable to drive an ultrasound probe 104. The ultrasound probe 104 may comprise a two-dimensional (2D) array of piezoelectric elements. The ultrasound probe 104 may comprise a group of transmit transducer elements 106 and a group of receive transducer elements 108, that normally constitute the same elements. In certain embodiment, the ultrasound probe 104 may be operable to acquire ultrasound image data covering at least a substantial portion of an anatomy, such as a lung, a fetus, a heart, a blood vessel, or any suitable anatomical structure.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or a plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to log-in to the ultrasound system 100, input patient data, image acquisition and scan parameters, settings, configuration parameters, select protocols and/or templates, change scan mode, manipulate tools for reviewing acquired ultrasound data, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, motion tracking, voice recognition, a mousing device, keyboard, camera and/or any other device capable of receiving a user directive. In certain embodiments, one or more of the user input devices 130 may be integrated into other components, such as the display system 134 or the ultrasound probe 104, for example. As an example, user input device 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, graphic processing units, microprocessors, microcontrollers, and/or the like. The signal processor 132 may be an integrated component, or may be distributed across various locations, for example. In an exemplary embodiment, the signal processor 132 may comprise an activity analysis processor 140, a workflow optimization processor 150, and a macro application processor 160 and may be capable of receiving input information from a user input device 130 and/or archive 138, generating an output displayable by a display system 134, and manipulating the output in response to input information from a user input device 130, among other things. The signal processor 132, activity analysis processor 140, workflow optimization processor 150, and macro application processor 160 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include an activity analysis processor 140 that comprises suitable logic, circuitry, interfaces and/or code that may be operable to analyze system activities during ultrasound examinations to recognize recurring system activity patterns over time. For example, ultrasound examinations may include one or more workflows, such as a patient intake workflow, imaging workflow, measurement workflow, analysis workflow, and/or any suitable workflow. The ultrasound system 100 often repeats many steps in performing the workflow(s) of an ultrasound examination. The activity analysis processor 140 may be configured to detect system activities during ultrasound examination workflow(s), such as preset selections, settings adjustments, selection of features presented at the display system 134, menu navigation inputs, manual image freeze inputs, automated image freeze inputs (e.g., in response to automated image recognition), input to begin a three-dimensional (3D) acquisition, image post-processing inputs, inputs to begin image segmentation; inputs to begin rendering; inputs to change a display mode, measurement inputs, inputs to anonymize images, inputs to save images (e.g., on a disk, in an archive, at cloud storage, etc.), inputs to generate and/or save reports, selections of different ultrasound probes 104, inputs to end an ultrasound examination, detection of ultrasound probe 104 placed in holder, detection of ultrasound system 100 inactivity, and/or any suitable system activity.

In various embodiments, the activity analysis processor 140 comprises suitable logic, circuitry, interfaces and/or code that may be operable to self-learn system activity patterns during workflow(s) of ultrasound examinations over time to detect recurring system activity patterns and suggest workflow modifications. For example, the activity analysis processor 140 may self-learn system activity patterns by executing pattern recognition algorithms, machine learning algorithms, artificial intelligence, and/or any suitable pattern recognition technique. As an example, the activity analysis processor 140 may deploy deep neural network(s) (e.g., artificial intelligence model(s)) that may be made up of, for example, an input layer, an output layer, and one or more hidden layers in between the input and output layers. Each of the layers may be made up of a plurality of processing nodes that may be referred to as neurons. For example, the activity analysis processor 140 may inference an artificial intelligence model comprising an input layer having a neuron for each detected system activity. The output layer may have neurons corresponding to one or more workflow modification recommendations. As an example, the output layer may provide a recommendation to modify preset settings, a recommendation to remove unused items presented at the display system 134, a recommendation to rearrange menu items to present most commonly used fields to a main menu or favorites screen, a recommendation to create a macro to automate a series of system activities in response to a detected triggering activity, and/or any suitable recommendation. Each neuron of each layer may perform a processing function and pass the processed system activity information to one of a plurality of neurons of a downstream layer for further processing. As an example, neurons of a first layer may learn to a sequence of system activities. The neurons of a second layer may learn to recognize a workflow corresponding to the sequence of system activities. The neurons of a third layer may learn to recognize recurrence of the sequence of system activities for the particular workflow of the ultrasound examination. The processing performed by the activity analysis processor 140 inferencing the deep neural network (e.g., convolutional neural network) may identify recurring system activity patterns and output corresponding workflow modification recommendations with a high degree of probability. The recommended workflow modifications may be provided to the workflow optimization processor 150, the macro application processor 160, and/or may be stored at archive 138 or any suitable data storage medium.

The signal processor 132 may include a workflow optimization processor 150 that comprises suitable logic, circuitry, interfaces and/or code that may be operable to present prompts at the display system 134 for accepting workflow modifications based on the recommended workflow modifications provided by the activity analysis processor 140. For example, the workflow optimization processor 150 may be configured to present a prompt at the display system 134 for modifying a workflow. The workflow optimization processor 150 may be configured to modify the workflow in response to receiving a user input accepting the workflow modification presented via the prompt at the display system 134. The workflow optimization processor 150 may be configured to present the prompt of the workflow modification based on the recurring system activity pattern detected by the activity analysis processor 140. As an example, the workflow optimization processor 150 may be configured to present a recommendation prompt to modify gain and contrast settings of a particular preset when the activity analysis processor 140 detects the user increases the gain and contrast settings each time the particular preset is selected. As another example, the workflow optimization processor 150 may be configured to present a recommendation prompt to remove unused items presented at the display system 134 when the activity analysis processor 140 detects an ultrasound system 100, or a particular user or group of users of an ultrasound system 100, is using only a portion of the features presented at the display system 134. As another example, the workflow optimization processor 150 may be configured to present a recommendation prompt to rearrange menu items to present most commonly used fields to a main menu or favorites screen presented at the display system 134 when the activity analysis processor 140 detects frequent user navigation of menus and sub-menus to access commonly used fields. As another example, the workflow optimization processor 150 may be configured to present a recommendation prompt to create a macro to automate a series of system activities in response to a detected triggering activity when the activity analysis processor 140 detects a series of system activity steps that are performed each time a particular system activity is performed.

The workflow optimization processor 150 may comprise suitable logic, circuitry, interfaces and/or code that may be operable to modify workflow items based on user acceptance of the recommended workflow modification presented via the prompt at the display system 134. For example, the workflow optimization processor 150 may be configured to store updated preset settings based on user acceptance of recommended preset setting modifications. As another example, the workflow optimization processor 150 may be configured to store updated display item and/or menu configuration settings in response to user acceptance of recommended unused display item removal and/or menu item rearrangement modifications. As another example, the workflow optimization processor 150 may be configured to instruct the macro application processor 160 to create and apply a macro to automate a series of system activities in response to a detected triggering activity based on user acceptance of the recommended macro.

The signal processor 132 may include a macro application processor 160 that comprises suitable logic, circuitry, interfaces and/or code that may be operable to create and apply a macro to automate a series of system activities in response to a detected triggering activity. For example, the macro application processor 160 may be configured to receive a detected recurring series of system activities from the activity analysis processor 140 and an instruction accepting the macro application from the workflow optimization processor 150. The macro application processor 160 may be configured to define a triggering activity and a series of system activities to automate in response to detection of the triggering activity. For example, the macro application processor 160 may define the automated image recognition of a fetal profile during a two-dimensional (2D) ultrasound scan as the triggering activity, which when detected automates the system activity steps of starting a three-dimensional (3D) acquisition, automatic detection of the fetal face in the 3D acquisition, removal of image data corresponding to excessive tissue adjacent the detected fetal face, starting image rendering, and changing from a standard display mode to a wide display mode so an ultrasound operator may present the rendered image to a patient. As another example, the macro application processor 160 may define the automated image recognition of a fetal brain during a 3D volume acquisition as the triggering activity, which when detected automates the system activity steps of starting a 3D brain segmentation algorithm, automatically performing measurements, and saving the measurements in a report. As another example, the macro application processor 160 may define the receiving of an “end exam” user input as the triggering activity, which when received automates the system activity steps of selecting images from an archive, anonymizing the selected images, and storing the anonymized images to a disk.

As another example, the macro application processor 160 may define the receiving of an “end exam” user input as the triggering activity, which when received automates the system activity steps of removing all archived cines acquired during the ultrasound examination retrospectively and sending single ultrasound images from the ultrasound examination to a cloud storage. As another example, the macro application processor 160 may define the receiving a selection of an endocavity ultrasound probe 104 as the triggering activity, which when received automates the system activity steps of entering a 3D acquisition mode, adjusting the acquisition parameters (e.g., volume size, line density, etc.) to values learned from previous sessions, and storing images to an archive when a user presses the freeze button. As another example, the macro application processor 160 may define the detection of the ultrasound probe 104 being placed in the holder and no activity performed by the ultrasound system 100 for two (2) minutes as the triggering activity, which when detected automates the system activity steps of anonymizing the ultrasound image data, storing the anonymized ultrasound image data in a custom DICOM format, and deleting the ultrasound image data from the ultrasound examination from the ultrasound system 100.

The macro application processor 160 may be configured to store the created macro in archive 138 and/or any suitable data storage medium. The macro application processor 160 may be configured to execute the appropriate macro based on detecting the defined triggering activity.

The display system 134 may be any device capable of communicating visual information to a user. For example, a display system 134 may include a liquid crystal display, a light emitting diode display, and/or any suitable display or displays. The display system 134 can be operable to present recommendation prompts, menus, sub-menus, menu fields, selectable feature items, selectable presets, preset settings, ultrasound images, image analysis tools, image measurement tools, selectable imaging modes, selectable ultrasound probes, and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores created macros, instructions for analyzing system activity during ultrasound examination workflow(s) and detecting recurring system activity patterns over time, instructions for recommending workflow modifications, instructions for executing approved workflow modifications, and instructions for executing macros in response to detected triggering activities, for example.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

Still referring to FIG. 1, the training system 200 may comprise a training engine 210 and a training database 220. The training engine 160 may comprise suitable logic, circuitry, interfaces and/or code that may be operable to train the neurons of the deep neural network(s) (e.g., artificial intelligence model(s)) inferenced (i.e., deployed) by the activity analysis processor 140. For example, the artificial intelligence model inferenced by the activity analysis processor 140 may be trained to automatically detect recurring system activity patterns and corresponding workflow modification recommendations. As an example, the training engine 210 may train the deep neural networks deployed by the activity analysis processor 140 using database(s) 220 of classified sequences of system activities. In various embodiments, the training engine 210 and/or training databases 220 may be remote system(s) communicatively coupled via a wired or wireless connection to the ultrasound system 100 as shown in FIG. 1. Additionally and/or alternatively, components or all of the training system 200 may be integrated with the ultrasound system 100 in various forms.

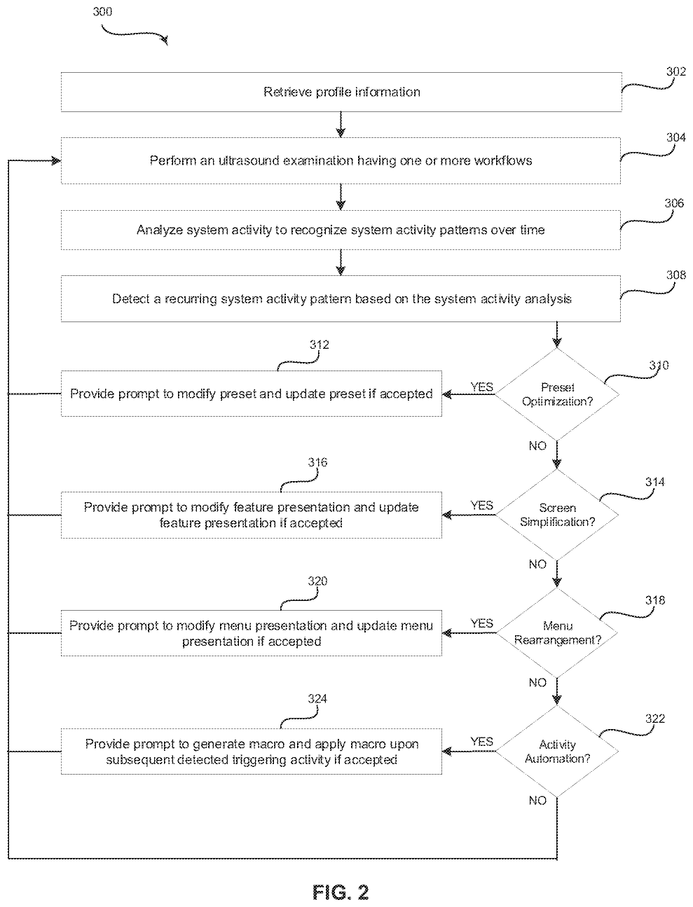

FIG. 2 is a flow chart 300 illustrating exemplary steps 302-324 that may be utilized for automatically recommending ultrasound examination workflow modifications based on detected activity patterns, in accordance with various embodiments. Referring to FIG. 2, there is shown a flow chart 300 comprising exemplary steps 302 through 324. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 302, a signal processor 132 of an ultrasound system 100 may retrieve profile information. For example, the signal processor 132 may retrieve an ultrasound system profile corresponding to a particular ultrasound system 100 when the ultrasound system is powered on. As another example, the signal processor 132 may retrieve a profile corresponding to a particular user or group of users in response to a user logging into the ultrasound system 100. The profile information may comprise display settings, selectable presets, preset settings, macros, and/or any suitable information. The signal processor 132 may apply the profile information during use of the ultrasound system 100, such as during one or more workflows of an ultrasound examination.

At step 304, the ultrasound system 100 may perform an ultrasound examination having one or more workflows. For example, an ultrasound operator may interact with the ultrasound system 100 to perform a patient intake workflow, imaging workflow, measurement workflow, analysis workflow, and/or any suitable workflow during an ultrasound examination.

At step 306, the signal processor 132 may analyze system activity to recognize system activity patterns over time. For example, an activity analysis processor 140 of the signal processor 132 may be configured to analyze system activities during ultrasound examinations to recognize recurring system activity patterns over time. The activity analysis processor 140 may be configured to analyze system activities during ultrasound examination workflow(s), such as preset selections, settings adjustments, selection of features presented at the display system 134, menu navigation inputs, manual image freeze inputs, automated image freeze inputs (e.g., in response to automated image recognition), input to begin a three-dimensional (3D) acquisition, image post-processing inputs, inputs to begin image segmentation; inputs to begin rendering; inputs to change a display mode, measurement inputs, inputs to anonymize images, inputs to save images (e.g., on a disk, in an archive, at cloud storage, etc.), inputs to generate and/or save reports, selections of different ultrasound probes 104, inputs to end an ultrasound examination, detection of ultrasound probe 104 placed in holder, detection of ultrasound system 100 inactivity, and/or any suitable system activity.

At step 308, the signal processor 132 may detect a recurring system activity pattern based on the system activity analysis. For example, the activity analysis processor 140 may be configured to self-learn system activity patterns during workflow(s) of ultrasound examinations over time to detect recurring system activity patterns and suggest workflow modifications. The activity analysis processor 140 may self-learn system activity patterns by executing pattern recognition algorithms, machine learning algorithms, artificial intelligence, and/or any suitable pattern recognition technique. The pattern recognition processing performed by the activity analysis processor 140 identifies recurring system activity patterns and output corresponding workflow modification recommendations.

At step 310, the signal processor 132 may determine whether a preset optimization is recommended based on the detected recurring system activity pattern. For example, a workflow optimization processor 150 of the signal processor 132 may receive workflow modification recommendations from the activity analysis processor 140. If the workflow modification recommendation is a preset optimization recommendation, the process proceeds to step 312. If the workflow modification recommendation is not a preset optimization recommendation, the process proceeds to step 314.

At step 312, the signal processor 132 may provide a prompt to modify a preset and update the preset if accepted. For example, a workflow optimization processor 150 of the signal processor 132 may be configured to present a recommendation prompt to modify particular settings (e.g., gain and contrast settings) of a specific preset when the activity analysis processor 140 detects the user modifies the particular settings each time the specific preset is selected. The recommendation prompt may include selectable options for accepting and/or rejecting the recommended workflow modification. The workflow optimization processor 150 may be configured to modify the preset in response to receiving a user input accepting the modification presented via the prompt at the display system 134. For example, the workflow optimization processor 150 may store the recommended setting to the preset at an archive 138 and/or any suitable data storage medium upon acceptance of the modification by a user via the user input device 130. In various embodiments, the modified preset may be stored in association with a particular user, group of users, or system profile. The process may return to step 304 to continue analyzing system activities.

At step 314, the signal processor 132 may determine whether a screen simplification is recommended based on the detected recurring system activity pattern. For example, the workflow optimization processor 150 of the signal processor 132 may receive workflow modification recommendations from the activity analysis processor 140. If the workflow modification recommendation is a screen simplification recommendation, the process proceeds to step 316. If the workflow modification recommendation is not a screen simplification recommendation, the process proceeds to step 318.

At step 316, the signal processor 132 may provide a prompt to modify feature presentation and update the feature presentation if accepted. For example, the workflow optimization processor 150 of the signal processor 132 may be configured to present a recommendation prompt to remove unused items presented at the display system 134 when the activity analysis processor 140 detects an ultrasound system 100, or a particular user or group of users of an ultrasound system 100, is using only a portion of the features presented at the display system 134. The workflow optimization processor 150 may be configured to modify the feature presentation at the display system 134 in response to receiving a user input accepting the modification presented via the prompt at the display system 134. For example, the workflow optimization processor 150 may store the recommended feature presentation display settings at an archive 138 and/or any suitable data storage medium upon acceptance of the modification by a user via the user input device 130. In various embodiments, the modified feature presentation display settings may be stored in association with a particular user, group of users, or system profile. The process may return to step 304 to continue analyzing system activities.

At step 318, the signal processor 132 may determine whether a menu rearrangement is recommended based on the detected recurring system activity pattern. For example, the workflow optimization processor 150 of the signal processor 132 may receive workflow modification recommendations from the activity analysis processor 140. If the workflow modification recommendation is a menu rearrangement recommendation, the process proceeds to step 320. If the workflow modification recommendation is not a menu rearrangement recommendation, the process proceeds to step 322.

At step 320, the signal processor 132 may provide a prompt to modify a menu presentation and update the menu presentation if accepted. For example, the workflow optimization processor 150 of the signal processor 132 may be configured to present a recommendation prompt to rearrange menu items to present most commonly used fields to a main menu or favorites screen presented at the display system 134 when the activity analysis processor 140 detects frequent user navigation of menus and sub-menus to access commonly used fields. The workflow optimization processor 150 may be configured to modify the menu arrangement at the display system 134 in response to receiving a user input accepting the modification presented via the prompt at the display system 134. For example, the workflow optimization processor 150 may store the recommended menu arrangement display settings at an archive 138 and/or any suitable data storage medium upon acceptance of the modification by a user via the user input device 130. In various embodiments, the modified menu arrangement display settings may be stored in association with a particular user, group of users, or system profile. The process may return to step 304 to continue analyzing system activities.

At step 322, the signal processor 132 may determine whether an activity automation is recommended based on the detected recurring system activity pattern. For example, the workflow optimization processor 150 of the signal processor 132 may receive workflow modification recommendations from the activity analysis processor 140. If the workflow modification recommendation is an activity automation recommendation, the process proceeds to step 324. If a workflow modification recommendation is not received, the process may return to step 304 to continue analyzing system activities.

At step 324, the signal processor 132 may provide a prompt to generate a macro and apply the macro upon subsequent detected triggering activity if accepted. For example, the workflow optimization processor 150 of the signal processor 132 may be configured to present a recommendation prompt to create a macro to automate a series of system activities in response to a detected triggering activity when the activity analysis processor 140 detects a series of system activity steps that are performed each time a particular system activity is performed. The workflow optimization processor 150 may be configured to instruct the macro application processor 160 of the signal processor 132 to create and apply a macro to automate a series of system activities in response to a detected triggering activity based on user acceptance of the recommended macro. The macro application processor 160 may be configured to create and apply a macro to automate a series of system activities in response to a detected triggering activity. For example, the macro application processor 160 may be configured to define a triggering activity and a series of system activities to automate in response to detection of the triggering activity. The macro application processor 160 may be configured to store the created macro in archive 138 and/or any suitable data storage medium. In various embodiments, the created macro may be stored in association with a particular user, group of users, or system profile. The macro application processor 160 may be configured to execute the appropriate macro based on detecting the defined triggering activity. The process may return to step 304 to continue analyzing system activities.

Aspects of the present disclosure provide a method 300 and system 100 for automatically recommending ultrasound examination workflow modifications based on detected activity patterns. In accordance with various embodiments, the method 300 may comprise performing 304, by an ultrasound system 100, an ultrasound examination having one or more workflows. The method 300 may comprise analyzing 306, by at least one processor 132, 140 of the ultrasound system 100, system activity detected during performance of the one or more workflows of the ultrasound examination. The method 300 may comprise detecting 308, by the at least one processor 132, 140, a recurring system activity pattern over a plurality of the ultrasound examination. The method 300 may comprise presenting 322, 324, by the at least one processor 132, 150 at a display system 134 and based on the detecting 308 the recurring system activity pattern, a prompt providing a recommendation to apply a macro to automate a series of system activities in response to detecting a triggering system activity. The method 300 may comprise receiving 324, by the at least one processor 132, 150, a user input accepting the recommendation to apply the macro. The method 300 may comprise applying 324, by the at least one processor 132, 160, the macro to automate the series of system activities in response to the detecting the triggering system activity.

In an exemplary embodiment, the method 300 may comprise retrieving 302 profile information. The profile information may be one of a user profile for a particular user retrieved in response to a user login, a group profile for a group of particular users retrieved in response to the user login, or a system profile corresponding with the ultrasound system 100. The method 300 may comprise storing 324 the macro in association with the profile information. In a representative embodiment, the method 300 may comprise generating 324, by the at least one processor 132, 160, the macro by defining the triggering system activity and the series of system activities to automate in response to the detecting the triggering system activity. In various embodiments, the system activity is one or more of a user interaction, a lack of user interaction, and an automated system action (e.g., an automatically detected image view, detecting an ultrasound probe 104 placed in a holder, etc.). In certain embodiments, the method 300 may comprise detecting 308, by the at least one processor 132, 140, a second recurring system activity pattern over the plurality of the ultrasound examination. The method 300 may comprise presenting 310, 312, by the at least one processor 132, 150 at the display system 134 and based on the detecting 308 the second recurring system activity pattern, a preset recommendation prompt providing a recommendation to modify particular settings of a preset when the second recurring system activity pattern is a recurring adjustment to the particular settings of the preset. The method 300 may comprise receiving 312, by the at least one processor 132, 150, a second user input accepting the recommendation to modify the particular settings of the preset. The method 300 may comprise modifying 312, by the at least one processor 132, 150, the particular settings of the preset. In an exemplary embodiment, the method 300 may comprise detecting 308, by the at least one processor 132, 140, a second recurring system activity pattern over the plurality of the ultrasound examination. The method 300 may comprise presenting 314, 316, by the at least one processor 132, 150 at the display system 134 and based on the detecting 308 the second recurring system activity pattern, a display simplification recommendation prompt providing a recommendation to remove unused items presented at the display system 134 when the second recurring system activity pattern is a pattern of lack of user interaction with the unused items. The method 300 may comprise receiving 316, by the at least one processor 132, 150, a second user input accepting the recommendation to remove the unused items presented at the display system 134. The method 300 may comprise modifying 316, by the at least one processor 132, 150, display settings to remove the unused items presented at the display system 134. In a representative embodiment, the method 300 may comprise detecting 308, by the at least one processor 132, 140, a second recurring system activity pattern over the plurality of the ultrasound examination. The method 300 may comprise presenting 318, 320, by the at least one processor 132, 150 at the display system 134 and based on the detecting the second recurring system activity pattern, a menu rearrangement recommendation prompt providing a recommendation to rearrange menu items to present commonly used fields to a main menu or favorites screen presented at the display system 134 when the second recurring system activity pattern is a pattern of user navigation of menus to access the commonly used fields. The method 300 may comprise receiving 320, by the at least one processor 132, 150, a second user input accepting the recommendation to rearrange menu items. The method 300 may comprise modifying 320, by the at least one processor 132, 150, display settings to rearrange the menu items to present the commonly used fields to the main menu or the favorites screen presented at the display system 134.

Various embodiments provide a system 100 for automatically recommending ultrasound examination workflow modifications based on detected activity patterns. The ultrasound system 100 may comprise at least one processor 132, 140, 150, 160 and a display system 134. The at least one processor 132, 140 may be configured to analyze system activity detected during performance of one or more workflows of an ultrasound examination. The at least one processor 132, 140 may be configured to detect a recurring system activity pattern over a plurality of the ultrasound examination. The at least one processor 132, 150 may be configured to present, at a display system 134 and based on the detecting the recurring system activity pattern, a prompt providing a recommendation to apply a macro to automate a series of system activities in response to detecting a triggering system activity. The at least one processor 132, 150 may be configured to receive a user input accepting the recommendation to apply the macro. The at least one processor 132, 160 may be configured to apply the macro to automate the series of system activities in response to the detecting the triggering system activity. The display system 134 may be configured to display the prompt providing the recommendation to apply the macro.

In a representative embodiment, the at least one processor 132 may be configured to retrieve profile information. The profile information may be one of a user profile for a particular user retrieved in response to a user login, a group profile for a group of particular users retrieved in response to the user login, or a system profile corresponding with the ultrasound system. The at least one processor 132, 160 may be configured to store the macro in association with the profile information. In various embodiments, the at least one processor 132, 160 may be configured to generate the macro by defining the triggering system activity and the series of system activities to automate in response to the detecting the triggering system activity. In certain embodiments, the system activity is one or more of a user interaction, a lack of user interaction, and an automated system action. In an exemplary embodiment, the at least one processor 132, 140 may be configured to detect a second recurring system activity pattern over the plurality of the ultrasound examination. The at least one processor 132, 150 may be configured to present, at the display system 134 and based on the detecting the second recurring system activity pattern, a preset recommendation prompt providing a recommendation to modify particular settings of a preset when the second recurring system activity pattern is a recurring adjustment to the particular settings of the preset. The at least one processor 132, 150 may be configured to receive a second user input accepting the recommendation to modify the particular settings of the preset. The at least one processor 132, 150 may be configured to modify the particular settings of the preset. The display system 134 may be configured to display the preset recommendation prompt providing the recommendation to modify the particular settings of the preset. In a representative embodiment, the at least one processor 132, 140 may be configured to detect a second recurring system activity pattern over the plurality of the ultrasound examination. The at least one processor 132, 150 may be configured to present, at the display system 134 and based on the detecting the second recurring system activity pattern, a display simplification recommendation prompt providing a recommendation to remove unused items presented at the display system 134 when the second recurring system activity pattern is a pattern of lack of user interaction with the unused items. The at least one processor 132, 150 may be configured to receive a second user input accepting the recommendation to remove the unused items presented at the display system 134. The at least one processor 132, 150 may be configured to modify display settings to remove the unused items presented at the display system 134. The display system 134 may be configured to display the display simplification recommendation prompt providing the recommendation to remove the unused items presented at the display system 134. In various embodiments, the at least one processor 132, 140 may be configured to detect a second recurring system activity pattern over the plurality of the ultrasound examination. The at least one processor 132, 150 may be configured to present, at the display system 134 and based on the detecting the second recurring system activity pattern, a menu rearrangement recommendation prompt providing a recommendation to rearrange menu items to present commonly used fields to a main menu or favorites screen presented at the display system 134 when the second recurring system activity pattern is a pattern of user navigation of menus to access the commonly used fields. The at least one processor 132, 150 may be configured to receive a second user input accepting the recommendation to rearrange the menu items. The at least one processor 132, 150 may be configured to modify display settings to rearrange the menu items to present the commonly used fields to the main menu or the favorites screen presented at the display system 134. The display system 134 may be configured to display the menu rearrangement recommendation prompt providing the recommendation to rearrange the menu items to present the commonly used fields to the main menu or the favorites screen.

Certain embodiments provide a non-transitory computer readable medium having stored thereon, a computer program having at least one code section. The at least one code section is executable by a machine for causing the machine to perform steps 300. The steps 300 may comprise analyzing 306 system activity detected during performance of the one or more workflows of an ultrasound examination. The steps 300 may comprise detecting 308 a recurring system activity pattern over a plurality of the ultrasound examination. The steps 300 may comprise presenting 322, 324, based on the detecting 308 the recurring system activity pattern, a prompt at a display system providing a recommendation to apply a macro to automate a series of system activities in response to detecting a triggering system activity. The steps 300 may comprise receiving 324 a user input accepting the recommendation to apply the macro. The steps 300 may comprise applying 324 the macro to automate the series of system activities in response to the detecting the triggering system activity.

In various embodiments, the steps 300 may comprise generating 324 the macro by defining the triggering system activity and the series of system activities to automate in response to the detecting the triggering system activity. In certain embodiments, the system activity is one or more of a user interaction, a lack of user interaction, and an automated system action. In an exemplary embodiment, the steps 300 may comprise detecting 308 a second recurring system activity pattern over the plurality of the ultrasound examination. The steps 300 may comprise presenting 310, 312, based on the detecting 308 the second recurring system activity pattern, a preset recommendation prompt at the display system 134 providing a recommendation to modify particular settings of a preset when the second recurring system activity pattern is a recurring adjustment to the particular settings of the preset. The steps 300 may comprise receiving 312 a second user input accepting the recommendation to modify the particular settings of the preset. The steps 300 may comprise modifying 312 the particular settings of the preset. In a representative embodiment, the steps 300 may comprise detecting 308 a second recurring system activity pattern over the plurality of the ultrasound examination. The steps 300 may comprise presenting 314, 316, based on the detecting 308 the second recurring system activity pattern, a display simplification recommendation prompt at the display system 134 providing a recommendation to remove unused items presented at the display system 134 when the second recurring system activity pattern is a pattern of lack of user interaction with the unused items. The steps 300 may comprise receiving 316 a second user input accepting the recommendation to remove the unused items presented at the display system 134. The steps 300 may comprise modifying 316 display settings to remove the unused items presented at the display system 134. In various embodiments, the steps 300 may comprise detecting 308 a second recurring system activity pattern over the plurality of the ultrasound examination. The steps 300 may comprise presenting 318, 320, based on the detecting 308 the second recurring system activity pattern, a menu rearrangement recommendation prompt at the display system 134 providing a recommendation to rearrange menu items to present commonly used fields to a main menu or favorites screen presented at the display system 134 when the second recurring system activity pattern is a pattern of user navigation of menus to access the commonly used fields. The steps 300 may comprise receiving 320 a second user input accepting the recommendation to rearrange menu items. The steps 300 may comprise modifying 320 display settings to rearrange the menu items to present the commonly used fields to the main menu or the favorites screen presented at the display system 134.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for automatically recommending ultrasound examination workflow modifications based on detected activity patterns.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method comprising:
performing, by an ultrasound system, an ultrasound examination having one or more workflows;
analyzing, by at least one processor of the ultrasound system, system activity detected during performance of the one or more workflows of the ultrasound examination;
detecting, by the at least one processor, a recurring system activity pattern over a plurality of the ultrasound examination;
presenting, by the at least one processor at a display system and based on the detecting the recurring system activity pattern, a prompt providing a recommendation to apply a macro to automate a series of system activities in response to detecting a triggering system activity;
receiving, by the at least one processor, a user input accepting the recommendation to apply the macro; and
applying, by the at least one processor, the macro to automate the series of system activities in response to the detecting the triggering system activity.

2. The method of claim 1, comprising:
retrieving profile information, wherein the profile information is one of a user profile for a particular user retrieved in response to a user login, a group profile for a group of particular users retrieved in response to the user login, or a system profile corresponding with the ultrasound system; and
storing the macro in association with the profile information.

3. The method of claim 1, comprising generating, by the at least one processor, the macro by defining the triggering system activity and the series of system activities to automate in response to the detecting the triggering system activity.

4. The method of claim 1, wherein the system activity is one or more of:
a user interaction,
a lack of user interaction, and
an automated system action.

5. The method of claim 1, comprising:
detecting, by the at least one processor, a second recurring system activity pattern over the plurality of the ultrasound examination;
presenting, by the at least one processor at the display system and based on the detecting the second recurring system activity pattern, a preset recommendation prompt providing a recommendation to modify particular settings of a preset when the second recurring system activity pattern is a recurring adjustment to the particular settings of the preset;
receiving, by the at least one processor, a second user input accepting the recommendation to modify the particular settings of the preset; and
modifying, by the at least one processor, the particular settings of the preset.

6. The method of claim 1, comprising:
detecting, by the at least one processor, a second recurring system activity pattern over the plurality of the ultrasound examination;
presenting, by the at least one processor at the display system and based on the detecting the second recurring system activity pattern, a display simplification recommendation prompt providing a recommendation to remove unused items presented at the display system when the second recurring system activity pattern is a pattern of lack of user interaction with the unused items;
receiving, by the at least one processor, a second user input accepting the recommendation to remove the unused items presented at the display system; and
modifying, by the at least one processor, display settings to remove the unused items presented at the display system.

7. The method of claim 1, comprising:
detecting, by the at least one processor, a second recurring system activity pattern over the plurality of the ultrasound examination;
presenting, by the at least one processor at the display system and based on the detecting the second recurring system activity pattern, a menu rearrangement recommendation prompt providing a recommendation to rearrange menu items to present commonly used fields to a main menu or favorites screen presented at the display system when the second recurring system activity pattern is a pattern of user navigation of menus to access the commonly used fields;
receiving, by the at least one processor, a second user input accepting the recommendation to rearrange menu items; and
modifying, by the at least one processor, display settings to rearrange the menu items to present the commonly used fields to the main menu or the favorites screen presented at the display system.

8. An ultrasound system comprising:
at least one processor configured to:
analyze system activity detected during performance of one or more workflows of an ultrasound examination;
detect a recurring system activity pattern over a plurality of the ultrasound examination;
present, at a display system and based on the detecting the recurring system activity pattern, a prompt providing a recommendation to apply a macro to automate a series of system activities in response to detecting a triggering system activity;
receive a user input accepting the recommendation to apply the macro; and
apply the macro to automate the series of system activities in response to the detecting the triggering system activity; and
a display system configured to display the prompt providing the recommendation to apply the macro.

9. The ultrasound system of claim 8, wherein the at least one processor is configured to:
retrieve profile information, wherein the profile information is one of a user profile for a particular user retrieved in response to a user login, a group profile for a group of particular users retrieved in response to the user login, or a system profile corresponding with the ultrasound system; and
store the macro in association with the profile information.

10. The ultrasound system of claim 8, wherein the at least one processor is configured to generate the macro by defining the triggering system activity and the series of system activities to automate in response to the detecting the triggering system activity.

11. The ultrasound system of claim 8, wherein the system activity is one or more of:
a user interaction,
a lack of user interaction, and
an automated system action.

12. The ultrasound system of claim 8, wherein:
the at least one processor is configured to:
detect a second recurring system activity pattern over the plurality of the ultrasound examination;
present, at the display system and based on the detecting the second recurring system activity pattern, a preset recommendation prompt providing a recommendation to modify particular settings of a preset when the second recurring system activity pattern is a recurring adjustment to the particular settings of the preset;
receive a second user input accepting the recommendation to modify the particular settings of the preset; and
modify the particular settings of the preset; and
the display system is configured to display the preset recommendation prompt providing the recommendation to modify the particular settings of the preset.

13. The ultrasound system of claim 8, wherein:
the at least one processor is configured to:
detect a second recurring system activity pattern over the plurality of the ultrasound examination;
present, at the display system and based on the detecting the second recurring system activity pattern, a display simplification recommendation prompt providing a recommendation to remove unused items presented at the display system when the second recurring system activity pattern is a pattern of lack of user interaction with the unused items;
receive a second user input accepting the recommendation to remove the unused items presented at the display system; and
modify display settings to remove the unused items presented at the display system; and
the display system is configured to display the display simplification recommendation prompt providing the recommendation to remove the unused items presented at the display system.

14. The ultrasound system of claim 8, wherein:
the at least one processor is configured to:
detect a second recurring system activity pattern over the plurality of the ultrasound examination;
present, at the display system and based on the detecting the second recurring system activity pattern, a menu rearrangement recommendation prompt providing a recommendation to rearrange menu items to present commonly used fields to a main menu or favorites screen presented at the display system when the second recurring system activity pattern is a pattern of user navigation of menus to access the commonly used fields;
receive a second user input accepting the recommendation to rearrange the menu items; and
modify display settings to rearrange the menu items to present the commonly used fields to the main menu or the favorites screen presented at the display system; and
the display system is configured to display the menu rearrangement recommendation prompt providing the recommendation to rearrange the menu items to present the commonly used fields to the main menu or the favorites screen.

15. A non-transitory computer readable medium having stored thereon, a computer program having at least one code section, the at least one code section being executable by a machine for causing the machine to perform steps comprising:
analyzing system activity detected during performance of the one or more workflows of an ultrasound examination;
detecting a recurring system activity pattern over a plurality of the ultrasound examination;
presenting, based on the detecting the recurring system activity pattern, a prompt at a display system providing a recommendation to apply a macro to automate a series of system activities in response to detecting a triggering system activity;
receiving a user input accepting the recommendation to apply the macro; and
applying the macro to automate the series of system activities in response to the detecting the triggering system activity.

16. The non-transitory computer readable medium of claim 15, comprising generating the macro by defining the triggering system activity and the series of system activities to automate in response to the detecting the triggering system activity.

17. The non-transitory computer readable medium of claim 15, wherein the system activity is one or more of:
a user interaction,
a lack of user interaction, and
an automated system action.

18. The non-transitory computer readable medium of claim 15, comprising:
detecting a second recurring system activity pattern over the plurality of the ultrasound examination;
presenting, based on the detecting the second recurring system activity pattern, a preset recommendation prompt at the display system providing a recommendation to modify particular settings of a preset when the second recurring system activity pattern is a recurring adjustment to the particular settings of the preset;
receiving a second user input accepting the recommendation to modify the particular settings of the preset; and
modifying the particular settings of the preset.

19. The non-transitory computer readable medium of claim 15, comprising:
detecting a second recurring system activity pattern over the plurality of the ultrasound examination;
presenting, based on the detecting the second recurring system activity pattern, a display simplification recommendation prompt at the display system providing a recommendation to remove unused items presented at the display system when the second recurring system activity pattern is a pattern of lack of user interaction with the unused items;
receiving a second user input accepting the recommendation to remove the unused items presented at the display system; and
modifying display settings to remove the unused items presented at the display system.

20. The non-transitory computer readable medium of claim 15, comprising:
detecting a second recurring system activity pattern over the plurality of the ultrasound examination;
presenting, based on the detecting the second recurring system activity pattern, a menu rearrangement recommendation prompt at the display system providing a recommendation to rearrange menu items to present commonly used fields to a main menu or favorites screen presented at the display system when the second recurring system activity pattern is a pattern of user navigation of menus to access the commonly used fields;
receiving a second user input accepting the recommendation to rearrange menu items; and
modifying display settings to rearrange the menu items to present the commonly used fields to the main menu or the favorites screen presented at the display system.
